(19) United States
(12) Patent Application Publication
(10) Pub. No.: US 2025/0066315 A1
(43) Pub. Date: Feb. 27, 2025

(54) NEO ACIDS AND DERIVATIVES THEREOF

(71) Applicants: Sunitha Sadula, Wilmington, DE (US); Erha Andini, Newark, DE (US); Dionisios G. Vlachos, Newark, DE (US)

(72) Inventors: Sunitha Sadula, Wilmington, DE (US); Erha Andini, Newark, DE (US); Dionisios G. Vlachos, Newark, DE (US)

(73) Assignee: University of Delaware, Newark, DE (US)

(21) Appl. No.: 18/718,861

(22) PCT Filed: Dec. 16, 2022
(86) PCT No.: PCT/US2022/053118

§ 371 Date: Jun. 12, 2024

Related U.S. Application Data
(60) Provisional application No. 63/291,186, filed on Dec. 17, 2021.

(51) Int. Cl. C07D 307/54; C07C 9/16; C07C 9/22; C07C 53/126; C07D 307/16

(52) U.S. Cl. CPC C07D 307/54; C07C 9/16; C07C 9/22; C07C 53/126; C07D 307/16

(57) ABSTRACT

The invention provides neo acids (NAs) and derivatives thereof. Also provided is a method for producing a NA, comprising hydrodeoxygenation (HDO) of a furan-containing neo acid (FNA) in the presence a metal triflate and a hydrogenation catalyst. The NA production method may further comprise hydroxyalkylation/alkylation (HAA) of 2-alkylfuran with levulinic acid or pyruvic acid in the presence of an acid catalyst, and at least one of 2-alkylfuran, levulinic acid and pyruvic acid may be from a renewable carbon source. The NA may have a bio-based content in the range of 20-100% according to, for example, ASTM-D6866. Further provided are methods for producing branched alkanes (BAs), tertrahydrofuran-containing neo acid (THFNA) and furan-containing neo ester (FNE) and side products such as iso acids (IAs).

### CROSS-REFERENCE TO RELATED APPLICATION

This application claims priority to U.S. Provisional Application No. 63/291,186, filed Dec. 17, 2021, and the contents of which are incorporated herein by reference in their entireties for all purposes.

### REFERENCE TO U.S. GOVERNMENT SUPPORT

This invention was made with government support under Grant No. DE-SC0001004 from the Department of Energy. The United States has certain rights in the invention.

### FIELD OF THE INVENTION

This invention relates generally to neo acids and derivatives thereof as well as their production, especially from renewable resources.

### BACKGROUND OF THE INVENTION

The rapid global population growth has increased the demand for food and other essential resources, which has resulted in intensified agriculture and industrial manufacturing activity. The large quantity of waste generated has become a growing issue, especially agricultural waste. A small amount of the waste becomes the raw materials for electricity generation and industrial applications. However, the rest is burnt or left to decompose in the field due to inefficient utilization and management practices, resulting in adverse environmental impacts. Recent advancement in the biomass waste conversion technologies has allowed for better utilization of the non-edible biomass as renewable sources of carbon to make valuable chemicals, such as fuels, lubricants, plastic, rubber, and detergents.

Neo acids, or highly branched carboxylic acids, are commercially valuable and currently produced from petroleum derived chemicals. The high steric hindrance provided by the neo acid structure imparts excellent thermal and hydrolytic stability, resistance to chemicals and oxidative compounds, and their low pour point allows for easy transportation, storage and handling. Depending on the chemistries applied to the neo acids (reduction, dehydration, esterification, etc.), various derivatives with diverse industrial applications are obtained.

Neo acids are commercially manufactured from petroleum derived olefins through Koch synthesis. However, the process involves harsh reaction conditions including high temperatures and pressures, the use of strong acids such as sulfuric acid, nitric acid, and hydrofluoric acid, and the process suffers from multi-step purifications to separate dimeric and trimeric isobutene by-products. There remains a need for a sustainable alternative to produce neo acids from the abundant and renewable resources such as plant biomass.

### SUMMARY OF THE INVENTION

The inventors have surprisingly discovered a strategy to synthesize neo acids from biomass-derived 2-alkylfuran and levulinic acid (LA) or pyruvic acid (PA) through two chemistries: C—C coupling of furans via hydroxyalkylation/alkylation (HAA) followed by ring-opening via hydrodeoxygenation (HDO) of furans.

A compound having a structure of any one of formulae 1-28 in Table 1 is provided.

TABLE 1
Compounds of formulae 1-28

| Formula # | Structure | |
|---|---|---|
| 1 | 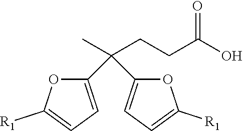 | R₁ is a C₂₋₁₈ alkyl group. |
| 2 | 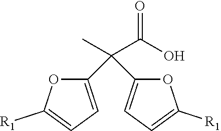 | R₁ is a C₁₋₁₈ alkyl group. |
| 3 | 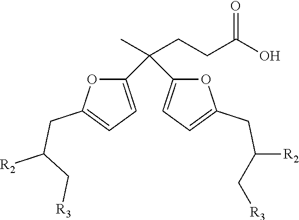 | R₂ and R₃ are independently selected from the group consisting of H and C₁₋₁₈ alkyl groups. |
| 4 | 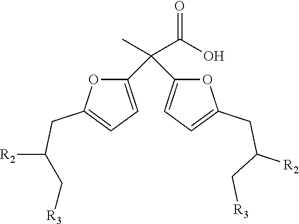 | R₂ and R₃ are independently selected from the group consisting of H and C₁₋₁₈ alkyl groups. |
| 5 | 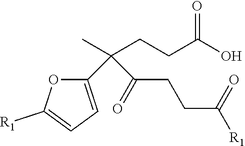 | R₁ is a C₁₋₁₈ alkyl group. |
| 6 | 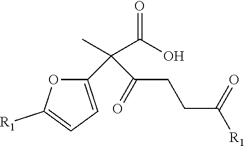 | R₁ is a C₁₋₁₈ alkyl group. |
| 7 | 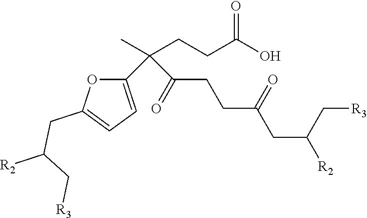 | R₂ and R₃ are independently selected from the group consisting of H and C₁₋₁₈ alkyl groups. |
| 8 | 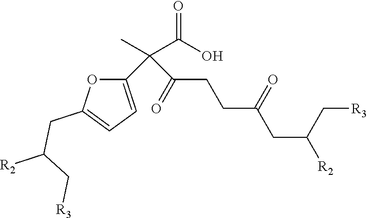 | R₂ and R₃ are independently selected from the group consisting of H and C₁₋₁₈ alkyl groups. |
| 9 | 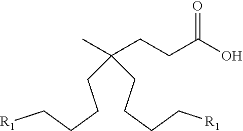 | R₁ is a C₁₋₁₈ alkyl group. |
| 10 | 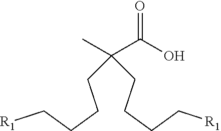 | R₁ is a C₁₋₁₈ alkyl group. |
| 11 | 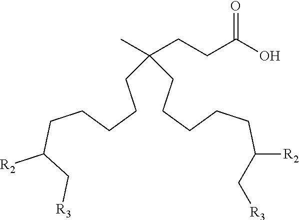 | R₂ and R₃ are independently selected from the group consisting of H and C₁₋₁₈ alkyl groups. |
| 12 | 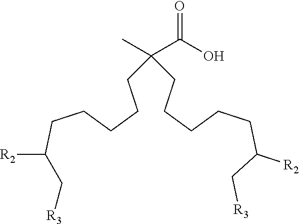 | R₂ and R₃ are independently selected from the group consisting of H and C₁₋₁₈ alkyl groups. |
| 13 | 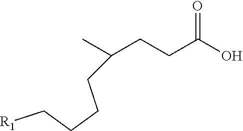 | R₁ is a C₁₋₁₈ alkyl group. |
| 14 | 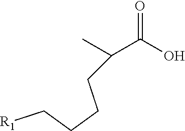 | R₁ is a C₁₋₁₈ alkyl group. |
| 15 | 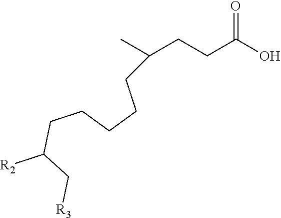 | R₂ and R₃ are independently selected from the group consisting of H and C₁₋₁₈ alkyl groups. |
| 16 | 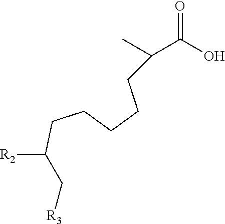 | R₂ and R₃ are independently selected from the group consisting of H and C₁₋₁₈ alkyl groups. |
| 17 | 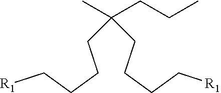 | R₁ is a C₁₋₁₈ alkyl group. |
| 18 | 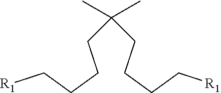 | R₁ is a C₁₋₁₈ alkyl group. |
| 19 | 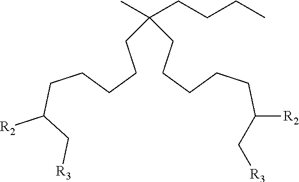 | R₂ and R₃ are independently selected from the group consisting of H and C₁₋₁₈ alkyl groups. |
| 20 | 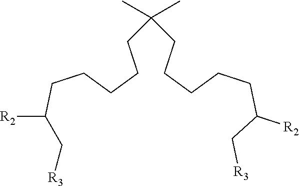 | R₂ and R₃ are independently selected from the group consisting of H and C₁₋₁₈ alkyl groups. |
| 21 | 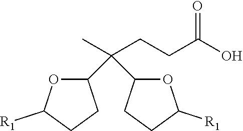 | R₁ is a C₁₋₁₈ alkyl group. |
| 22 | 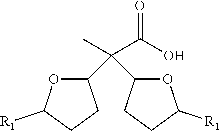 | R₁ is a C₁₋₁₈ alkyl group. |
| 23 | 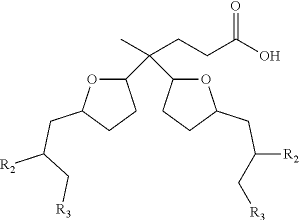 | R₂ and R₃ are independently selected from the group consisting of H and C₁₋₁₈ alkyl groups. |
| 24 | 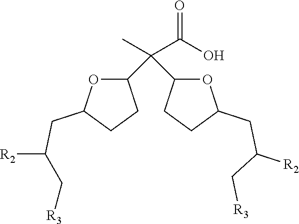 | R₂ and R₃ are independently selected from the group consisting of H and C₁₋₁₈ alkyl groups. |
| 25 | 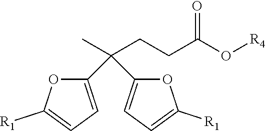 | R₁ is H or a C₁₋₁₈ alkyl group. R₄ is a C₃₋₁₈ alkyl group. |
| 26 | 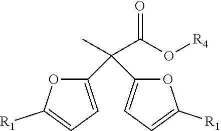 | R₁ and R₄ are independently a C₁₋₁₈ alkyl group. |
| 27 | 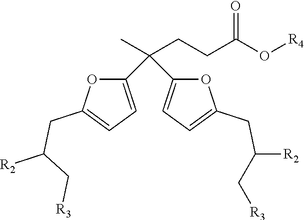 | R₂ and R₃ are independently selected from the group consisting of H and a C₁₋₁₈ alkyl group. R₄ is a C₃₋₁₈ alkyl group. |
| 28 | 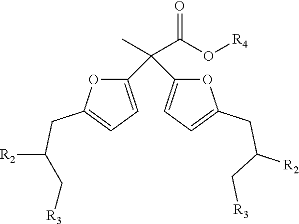 | R₂ and R₃ are independently selected from the group consisting of H and a C₁₋₁₈ alkyl group. R₄ is a C₃₋₁₈ alkyl group. |

The compound may be a furan-containing neo acid (FNA) having a structure of any one of formulae 1-4.

The compound may be a furan-containing neo acid ring opening (FNA RO) having a structure of any one of formulae 5-8.

The compound may be a neo acid (NA) having a structure of any one of formulae 9-12.

The compound may be an iso acid (IA) having a structure of any one of formulae 13-16.

The compound may be a branched alkane (BA) having a structure of any one of formulae 17-20.

The compound may be a tertrahydrofuran-containing neo acid (THFNA) having a structure of any one of formulae 21-24.

The compound may be a furan-containing neo ester (FNE) having a structure of any one of formulae 25-28.

A method for producing a neo acid (NA) is provided. The NA production method comprises hydrodeoxygenation (HDO) of a furan-containing neo acid (FNA) in the presence of a metal triflate and a hydrogenation catalyst. The NA production method may further comprise hydroxyalkylation/alkylation (HAA) of 2-alkylfuran with levulinic acid or pyruvic acid in the presence of an acid catalyst. At least one of 2-alkylfuran, levulinic acid and pyruvic acid may be from a renewable carbon source. The NA may have a structure of any one of formulae 9-12. The FNA may have a structure of any one of formulae 1-4.

A method for producing a branched alkane (BA) is provided. The BA production method comprises hydrodeoxygenation (HDO) of a furan-containing neo acid (FNA) in the presence of a solid acid supported metal-metal oxide catalyst or a physical mixture of a metal-based catalyst with a solid acid. The BA production method may further comprise hydroxyalkylation/alkylation (HAA) of 2-alkylfuran with levulinic acid or pyruvic acid in the presence of an acid catalyst to produce the FNA. At least one of 2-alkylfuran, levulinic acid and pyruvic acid may be from a renewable carbon source. The BA may have a structure of any one of formulae 17-20. The FNA may have a structure of any one of formulae 1-4.

A method for producing a tetrahydrofuran-containing neo acid (THFNA) is provided. The THFNA production method comprises hydrogenation of a furan-containing neo acid (FNA) in the presence of a hydrogenation catalyst. The THFNA production method may further comprise hydroxyalkylation/alkylation (HAA) of 2-alkylfuran with levulinic acid or pyruvic acid in the presence of an acid catalyst to produce the FNA. At least one of 2-alkylfuran, levulinic acid and pyruvic acid may be from a renewable carbon source. The THFNA may have a structure of any one of formulae 21-24. The FNA may have a structure of any one of formulae 1-4.

A method for producing a furan-containing neo ester (FNE) is provided. The FNE production method comprises esterification of the FNA with an alcohol. The FNE production method may further comprise hydroxyalkylation/alkylation (HAA) of 2-alkylfuran with levulinic acid or pyruvic acid in the presence of an acid catalyst to produce the FNA. At least one of 2-alkylfuran, levulinic acid and pyruvic acid may be from a renewable carbon source. The FNE may have a structure of any one of formulae 25-28. The FNA may have a structure of any one of formulae 1-4.

### BRIEF DESCRIPTION OF THE DRAWINGS

FIG. 1 is Scheme 1 showing synthesis of bio-based neo acids from biomass-derived 2-alkylfuran and levulinic acid or pyruvic acid.

FIG. 2 is Scheme 2 showing examples of derivatives synthesized from FNA, where FNA is synthesized from biomass-derived 2-alkylfuran and levulinic acid or pyruvic acid.

FIGS. 3A-B illustrate synthesis of furan, 2-methylfuran, 2-alkylfuran, levulinic acid, and pyruvic acid from biomass.

FIG. 4 is a diagram showing diverse exemplary industrial applications of neo acids and their derivatives.

FIG. 5 shows self-condensation products and ring opening products from the HAA reaction of 2-pentylfuran and levulinic acid over an acid catalyst.

FIG. 6 shows yields of FNA and FNA RO in the HAA reaction of 2-pentylfuran and levulinic acid over various acid catalysts. Reaction conditions: 20 mmol 2-pentylfuran (2-PF), 10 mmol levulinic acid (LA), 0.107 mmol H⁺ catalyst, 65° C., 6 h, 800 rpm.

FIGS. 7A-C show the results for HAA reaction multi parameter optimization using NEXTorch. A) Optimal FNA+FNA RO Yield (%) obtained in experiments in each iteration, where the error bar indicates the standard deviation of the yield from 2 measurements; B) Principal component analysis of the correlations between reaction conditions and outcomes; and C) Correlation matrix between reaction conditions and outcomes. Statistical Method: Latin Hypercube (16 initial sampling point, iteration 0). NEXTorch generated 4 subsequent sampling points per iterations.

FIGS. 8A-B show recyclability of Aquivion PW79S acid catalyst for the HAA reaction of 2-pentylfuran and levulinic acid to produce FNA. Reaction conditions: 20 mmol 2-pentylfuran (2-PF), 10 mmol levulinic acid (LA), 0.107 mmol H+ catalyst, 65° C., 6 h, 800 rpm. A) Percentage of conversion of 2-PF and LA, and yields of FNA, FNA RO, and total carbon balance after 1-5 cycles. B) FT-IR Spectra before (fresh) and after 2 cycles of the HAA reaction. The catalyst changed from white powder (bottom image) before (fresh) to dark brown powder (top image) after 2 cycles of the HAA reaction.

FIG. 9 shows neo acid and iso acid product yield from the HDO reaction of FNA and FNA RO during metal triflate screening in presence of a hydrogenation catalyst. Reaction conditions: 1 mmol FNA, 6 mol % Al(OTf)₃, 2 mol % Pd/C, 10 mL n-octane, 30 bar H2, 180° C., 1 h, 500 rpm. Conversion ≥99% & total carbon balance≤70%.

FIG. 10 shows neo acid and iso acid product yield from the HDO reaction of FNA and FNA RO over different hydrogenation catalysts. Reaction conditions: 1 mmol FNA, 6 mol % Al(OTf)₃, 2 mol % hydrogenation catalyst, 10 mL n-octane, 30 bar H₂, 180° C., 1 h, 500 rpm. Conversion ≥99% & total carbon balance≤70%.

FIG. 11 shows neo acid and iso acid product yield from the HDO reaction of FNA and FNA RO in different organic solvents. Reaction conditions: 1 mmol FNA, 6 mol % Al(OTf)₃, 2 mol % Pd/C, 10 mL organic solvent, 30 bar H₂, 180° C., 1 h, 500 rpm.

FIGS. 12A-D show neo acid and iso acid product yield at various A) catalyst molar ratio, B) H₂ pressure, C) reaction temperature, and D) reaction time. Reaction conditions: 1 mmol FNA, 6 mol % Al(OTf)₃, 2 mol % Pd/C, 10 mL cyclohexane, 500 rpm.

FIGS. 13A-D show GC overlay for the HDO product mixture obtained with and without a catalyst. A) HDO reaction over Al(OTf) 3+Pd/C catalysts, B) HDO reaction over Al(OTf) 3 only (without hydrogenation catalyst), C) HDO reaction over Pd/C only (without metal triflate), D) HDO reaction without any hydrogenation catalyst and metal triflate (Blank reaction). Reaction conditions: 1 mmol FNA, 6 mol % Al(OTf)₃, 2 mol % Pd/C, 10 mL cyclohexane, 30 bar H₂, 180° C., 1 h, 500 rpm.

FIG. 14 is Scheme 4 showing an example of esterification of FNA with alcohol (methanol) making FNE.

FIG. 15 is Scheme 5 showing an example of hydrogenation of FNA making THFNA.

FIG. 16 is Scheme 6 showing an example of hydrodeoxygenation of FNA making BA.

### DETAILED DESCRIPTION OF THE INVENTION

The present invention provides novel strategies to produce neo acids (NAs) with tailored molecular architecture from biomass, as well as other derivatives including branched alkanes (BAs), furan-containing neo esters (FNEs), and tetrahydrofuran-containing neo acids (THFNAs) and side products such as iso acids (IAs). The invention is based on the discovery by the inventors of a synthesis pathway of bio-based neo acids from biomass-derived 2-alkylfuran and levulinic acid or pyruvic acid comprised of hydroxyalkylation/alkylation (HAA) using an acid catalyst followed by hydrodeoxygenation (HDO) using metal triflate and Pd/C. While the HAA chemistry has been reported to increase carbon number of biomass-derived platform molecules and the HDO chemistry has been reported to ring-open aromatic functionalities to make jet fuels and diesel from biomass, the inventors have surprisingly discovered the use of these two chemistries to make bio-based neo acid with tailored molecular architecture. In general, the neo acids with tailored molecular architecture are produced from reacting 2-alkylfurans with levulinic acid or pyruvic acid. In a preferred embodiment, at least one of the 2-alkylfuran, levulinic acid and pyruvic acid is obtained from a renewable carbon source. In addition, the inventors have demonstrated the feasibility of converting an FNA precursor into other derivatives, such as branched alkanes (BAs) through HDO over Ir—ReOₓ/SiO₂, tetrahydrofuran-containing neo acids (THFNAs) through hydrogenation over Pd/C, and furan-containing neo esters (FNEs) through esterification with an alcohol. The inventors have successfully implemented the HAA, HDO, hydrogenation, and esterification chemistries to make bio-based BAs, THFNAs, and FNEs with tailored molecular architecture.

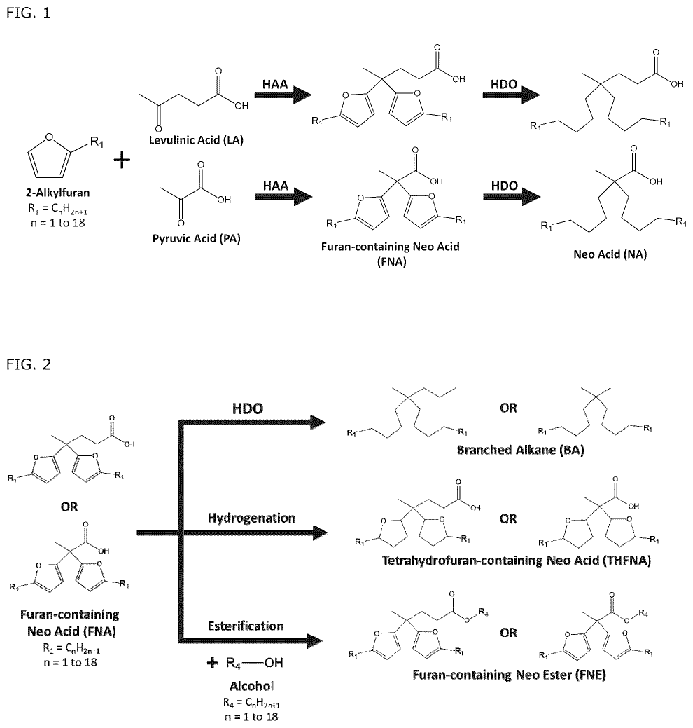

The inventors have demonstrated a strategy to synthesize renewable neo acids from biomass-derived 2-alkylfuran and levulinic acid (LA) or pyruvic acid (PA) through two chemistries: C—C coupling of furans via hydroxyalkylation/alkylation (HAA) followed by ring-opening via hydrodeoxygenation (HDO) of furans (Scheme 1) (FIG. 1). These chemistries have been extensively investigated to increase the carbon chain of sugars and produce jet fuels, diesel, and lubricant base oils (Liu et al., Science advances, 5(2), p.eaav5487 (2019); Corma et al., Energy & Environmental Science, 5(4), 6328-6344 (2012); Sutton et al., Nature chemistry, 5(5), 428-432 (2013); Li et al., Green Chemistry, 20(2), 362-368 (2018)). However, their adaptation to make neo acids with tailored architecture has not yet been reported. The HAA reaction between 2-pentylfuran (2-PF) and levulinic acid (LA) show successful conversion of the reactants into neo-acid precursor FNA over a solid acid catalyst, yielding an optimum yield of 90% for FNA and its ring-opening product (FNA RO). During the HDO reaction, the FNA RO product also led to the formation of a desired neo acid. However, complete conversion of LA was not achieved due to the participation of 2-PF in a self-condensation side reaction facilitated by the water produced during the condensation reaction. The selective HDO of the FNA furan ring without reducing the carboxylic acid functionality was a challenge to make a neo acid. Upon catalyst and solvent screenings, and parameter optimization studies for the HDO step, the inventors have surprisingly achieved a 40% yield of the desired neo acid (C₂₃-NA) with 15% of iso acid (IA), a cracked product, with nearly 30% missing total carbon balance. The missing total carbon balance could be due to the formation of high molecular weight products that were not eluted and detected by the GC/GC-MS.

In addition, the inventors have shown the feasibility of converting the FNA into other derivatives, such as branched alkanes (BAs) through hydrodeoxygenation over a different catalyst, tetrahydrofuran-containing neo acids (THFNAs) through hydrogenation, and furan-containing neo esters (FNEs) through esterification with an alcohol (Scheme 2) (FIG. 2).

The terms “hydrodeoxygenation (HDO)” and “hydrodeoxygenation (HDO) reaction” are used herein interchangeably and refer to a chemical reaction in which a carbon oxygen single bond undergoes cleavage of a C—O bond by a hydrogen and the oxygen atom is removed from the compound, typically, in the presence of a catalyst.

The terms “hydrogenation” and “hydrogenation reaction” are used interchangeably herein and refer to a chemical reaction between a molecular hydrogen and a compound resulting a reduced or saturated organic compound, typically, in the presence of a catalyst.

The terms “biomass” and “biomass waste” as used herein interchangeably and refer to forestry residues and agricultural wastes, such as corn stover, tree branches, wheat straws, and the like.

The term “renewable carbon source” as used herein refers to biomass from which a renewable carbon atom may be derived. The biomass may be a plant or other renewable agricultural or forestry material. Fossil fuel and petroleum are not renewable carbon source, and a carbon atom derived from fossil fuel or petroleum is not a renewable carbon atom.

The term “bio-based content” as used herein refers to a percentage of renewable carbon atoms relative to all the carbon atoms in a compound. The bio-based content of a compound or composition may be measured any conventional process known in the art, for example, ASTM-D6866.

ASTM-D6866 is a standard test method to determine biobased carbon/biogenic carbon content of solid, liquid, and gaseous samples using radiocarbon analysis. It is performed by deriving a ratio of the amount of radiocarbon (carbon 14) in an unknown sample to that of a modern reference standard. The ratio may be reported as a percentage with the unit “pMC” (i.e., percent modern carbon). If the material being analyzed is a mixture of present-day radiocarbon and fossil carbon that contains no radiocarbon, then the pMC value obtained correlates directly to the amount of biomass material present in the sample. Combining fossil carbon with present-day carbon into a material may result in a dilution of the present-day pMC content. A bio-mass content result may be derived by assigning 100% equal to 107.5 pMC and 0% equal to 0 pMC. In this regard, a sample measuring 99 pMC gives an equivalent bio-based content result of 93% and if a sample is diluted with 50% petroleum derivative the pMC value of 54.

According to one aspect of the present invention, a compound such as FNA, FNA RO, NA, or IA may have a bio-based content in the range of about 20-100%, 30-100%, 40-100%, 50-100%, 60-100%, 70-100%, 80-100%, 90-100%, 20-90%, 30-90%, 40-90%, 50-90%, 60-90%, 70-90% or 80-90%; or at least about 20%, 30%, 35%, 40%, 45%, 50%, 55%, 60%, 65%, 70%, 75%, 80%, 85%, 90%, 95%, 96%, 97%, 98%, or 99%, or 100%; preferably about 40-100%; and most preferably about 50-100%, as determined according to ASTM-D6866.

The terms “biomass waste-derived”, “biomass-derived”, “bio-derived” and “bio-based” as used herein interchangeably and refer to a compound that is obtained from a renewable carbon resource such as plants and contains either solely or substantially renewable carbon atoms with no or a very minimal amount of carbon atoms derived from fossil fuel or petroleum. For example, a biomass-derived compound may have more than 50%, 60%, 70%, 80%, 90%, 95% or 99%, or 100% of renewable carbon atoms, based on total number of carbon atoms in the compound.

The term “biomass waste-derived 2-alkylfuran” as used herein refers to 2-alkylfuran that is derived from a renewable carbon source and contains either sole or substantially renewable carbon, and no or a very minimal amount of fossil fuel-based or petroleum-based carbon.

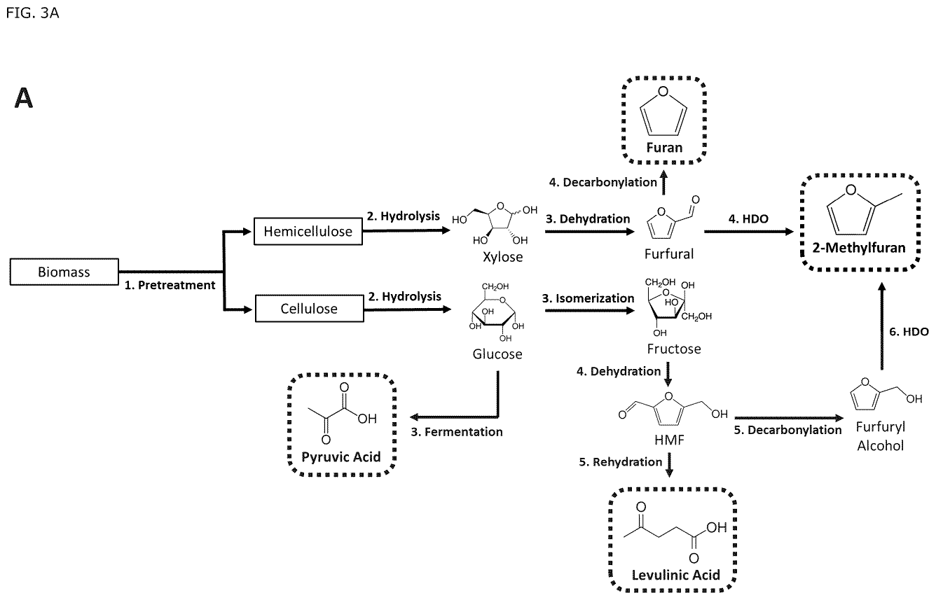

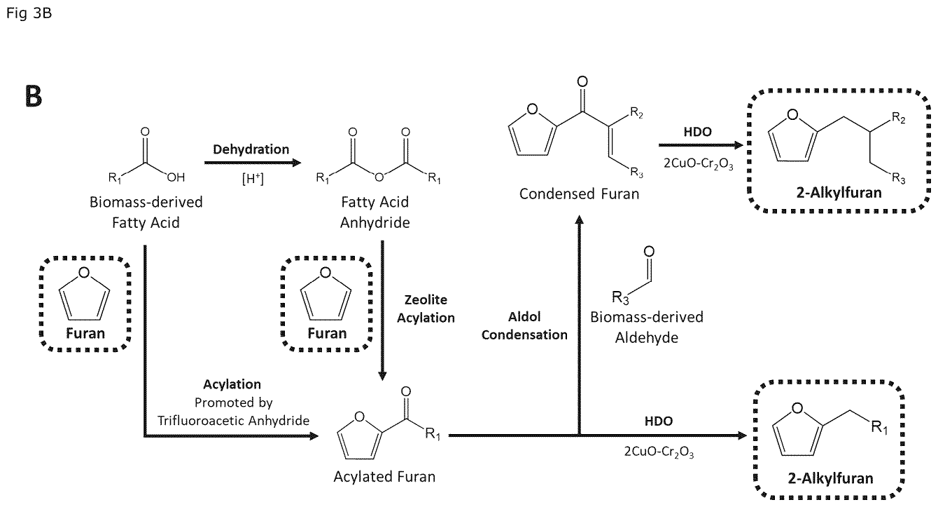

The 2-alkylfuran or a mixture of 2-alkylfurans may be prepared by a process comprising dehydration and hydrodeoxygenation (HDO) of biomass-derived C₅ sugars (FIG. 3A), and acylation of biomass-derived furan with one or more carboxylic acids or carboxylic acid anhydrides followed by the HDO reaction (FIG. 3B). The synthesis of levulinic acid and pyruvic acid from biomass is also shown in FIG. 3A. The synthesis of alcohol of different carbon lengths from biomass may be achieved through fermentation, the Guerbet reaction, or fatty alcohols (C₈-C₁₈) obtained directly from natural fats and oils.

The present invention provides neo acids and derivatives thereof, for example, compounds having a structure of any one of formulae 1-28 (Table 1). R₁ may be butyl, pentyl, hexyl, heptyl, octyl, dodecyl, octadecanyl, cyclopentyl, or cyclohexyl. R₁ may have the formula: —(CHR₂)—CH₂R₃). R₂ and R₃ may be independently H or a linear or branched alkyl group having 2 to 18 carbon atoms. R₂ may be methyl, butyl, hexyl, dodecyl, cyclopentyl, or cyclohexyl. R₃ may be methyl, butyl, hexyl, dodecyl, cyclopentyl, or cyclohexyl. Suitable examples of branched alkyl groups, that is having one or more branches, include methylpropyl, methylbutyl, methyldodecyl, ethylpropyl, ethyloctyl, and cyclopentyheptyl. Suitable examples of biomass-derived alcohols to use for the preparation of FNE include ethanol, butanol, hexanol, isopropanol, and dodecanol. Such biomass-derived alcohols may be derived from any suitable biomass, including corn grain, soya bean grain, any kind of hard wood, any kind of soft wood, and algae. The synthesis of alcohol of different carbon lengths from biomass may be achieved through fermentation, the Guerbet reaction, or fatty alcohols (C₈-C₁₈) obtained directly from natural fats and oils.

According to Scheme 1, the furan-containing neo acid (FNA) may have a structure of any one of formulae 1-4; The furan-containing neo acid ring opening (FNA RO) may have a structure of any one of formulae 5-8; and the neo acid (NA) may have a structure of any one of formulae 9-12. A side product in the reaction according to Scheme 1 may be an iso acid (IA). The IA may have a structure of any one of formulae 13-16. For formula 1, the C₂₋₁₈ alkyl group may be a substituted or an unsubstituted, a cyclic or acyclic, or a branched or an unbranched alkyl group having 2 to 18 carbon atoms. For each of formulae 2-16, the C₁₋₁₈ alkyl group may be a substituted or an unsubstituted, a cyclic or acyclic, or a branched or an unbranched alkyl group having 1 to 18 carbon atoms.

Where the FNA has the structure of formula 1, the FNA may be 4,4-bis(5-ethylfuran-2-yl)pentanoic acid where R₁ is C₂H₅; 4,4-bis(5-propylfuran-2-yl)pentanoic acid where R₁ is C₃H₇, 4,4-bis(5-butylfuran-2-yl)pentanoic acid where R₁ is C₄H₉; 4,4-bis(5-pentylfuran-2-yl)pentanoic acid where R₁ is C₅H₁₁; 4,4-bis(5-hexylfuran-2-yl)pentanoic acid where R₁ is C₆H₁₃; or 4,4-bis(5-heptylfuran-2-yl)pentanoic acid where R₁ is C₇H₁₅.

Where the FNA has the structure of formula 2, the FNA may be 2,2-bis(5-methylfuran-2-yl)propanoic acid where R₁ is CH₃; 2,2-bis(5-ethylfuran-2-yl)propanoic acid where R₁ is C₂H₅; 2,2-bis(5-propylfuran-2-yl)propanoic acid where R₁ is C₃H₇; 2,2-bis(5-butylfuran-2-yl)propanoic acid where R₁ is C₄H₉; 2,2-bis(5-pentylfuran-2-yl)propanoic acid where R₁ is C₅H₁₁; 2,2-bis(5-hexylfuran-2-yl)propanoic acid where R₁ is C₆H₁₃; or 2,2-bis(5-heptylfuran-2-yl)propanoic acid where R₁ is C₇H₁₅.

Where the FNA RO has the structure of formula 5, the FNA RO may be 4-methyl-4-(5-methylfuran-2-yl)-5,8-dioxononanoic acid where R₁ is CH₃; 4-(5-ethylfuran-2-yl)-4-methyl-5,8-dioxodecanoic acid where R₁ is C₂H₅; 4-methyl-5,8-dioxo-4-(5-propylfuran-2-yl)undecanoic acid where R₁ is C₃H₇; 4-(5-butylfuran-2-yl)-4-methyl-5,8-dioxododecanoic acid where R₁ is C₄H₉; 4-methyl-5,8-dioxo-4-(5-pentylfuran-2-yl)tridecanoic acid where R₁ is C₅H₁₁; 4-(5-hexylfuran-2-yl)-4-methyl-5,8-dioxotetradecanoic acid where R₁ is C₆H₁₃; or 4-(5-heptylfuran-2-yl)-4-methyl-5,8-dioxopentadecanoic acid where R₁ is C₇H₁₅.

Where the FNA RO has the structure of formula 6, the FNA RO may be 2-methyl-2-(5-methylfuran-2-yl)-3,6-dioxoheptanoic acid where R₁ is CH₃; 2-(5-ethylfuran-2-yl)-2-methyl-3,6-dioxooctanoic acid where R₁ is C₂H₅; 2-methyl-3,6-dioxo-2-(5-propylfuran-2-yl)nonanoic acid where R₁ is C₃H₇; 2-(5-butylfuran-2-yl)-2-methyl-3,6-dioxodecanoic acid where R₁ is C₄H₉; 2-methyl-3,6-dioxo-2-(5-pentylfuran-2-yl)undecanoic acid where R₁ is C₅H₁₁; 2-(5-hexylfuran-2-yl)-2-methyl-3,6-dioxododecanoic acid where R₁ is C₆H₁₃; or 2-(5-heptylfuran-2-yl)-2-methyl-3,6-dioxotridecanoic acid where R₁ is C₇H₁₅.

Where the NA has the structure of formula 9, the NA may be 4-methyl-4-pentylnonanoic acid where R₁ is CH₃; 4-hexyl-4-methyldecanoic acid where R₁ is C₂H₅; 4-heptyl-4-methylundecanoic acid where R₁ is C₃H₇; 4-methyl-4-octyldodecanoic acid where R₁ is C₄H₉; 4-methyl-4-nonyltridecanoic acid where R₁ is C₅H₁₁; 4-decyl-4-methyltetradecanoic acid where R₁ is C₆H₁₃; or 4-methyl-4-undecylpentadecanoic acid where R₁ is C₇H₁₅.

Where the NA has the structure of formula 10, the NA may be 2-methyl-2-pentylheptanoic acid where R₁ is CH₃; 2-hexyl-2-methyloctanoic acid where R₁ is C₂H₅; 2-heptyl-2-methylnonanoic acid where R₁ is C₃H₇; 2-methyl-2-octyldecanoic acid where R₁ is C₄H₉; 2-methyl-2-nonylundecanoic acid where R₁ is C₅H₁₁; 2-decyl-2-methyldodecanoic acid where R₁ is C₆H₁₃; or 2-methyl-2-undecyltridecanoic acid where R₁ is C₇H₁₅.

Where the IA has the structure of formula 13, the IA may be 4-methylnonanoic acid where R₁ is CH₃; 4-methyloctanoic acid where R₁ is C₂H₅; 4-methylnonanoic acid where R₁ is C₃H₇; 4-methyldecanoic acid where R₁ is C₄H₉; 4-methyltridecanoic acid where R₁ is C₅H₁₁; 4-methyltetradecanoic acid where R₁ is C₆H₁₃; or 4-methylpentadecanoic acid where R₁ is C₇His.

Where the IA has the structure of formula 14, the IA may be 2-methylheptanoic acid where R₁ is CH₃; 2-methyldecanoic acid where R₁ is C₂H₅; 2-methylundecanoic acid where R₁ is C₃H₇; 2-methyldecanoic acid where R₁ is C₄H₉; 2-methylundecanoic acid where R₁ is C₅H₁₁; 2-methyldodecanoic acid where R₁ is C₆H₁₃; or 2-methyltridecanoic acid where R₁ is C₇His.

According to Scheme 2 (FIG. 2), the branched alkane (BA) may have a structure of any one of formulae 17-20; the tertrahydrofuran-containing neo acid (THFNA) may have a structure of any one of formulae 21-24; and the furan-containing neo ester (FNE) may have a structure of any one of formulae 25-28.

For each of formulae 17-24 and 26, the C₁-18 alkyl group may be a substituted or an unsubstituted, a cyclic or acyclic, or a branched or an unbranched alkyl group having 1 to 18 carbon atoms.

For formula 25, 27 or 28, the C₁₋₁₈ alkyl group may be a substituted or an unsubstituted, a cyclic or acyclic, or a branched or an unbranched alkyl group having 1 to 18 carbon atoms, and the C₃₋₁₈ alkyl group may be a substituted or an unsubstituted, a cyclic or acyclic, or a branched or an unbranched alkyl group having 3 to 18 carbon atoms.

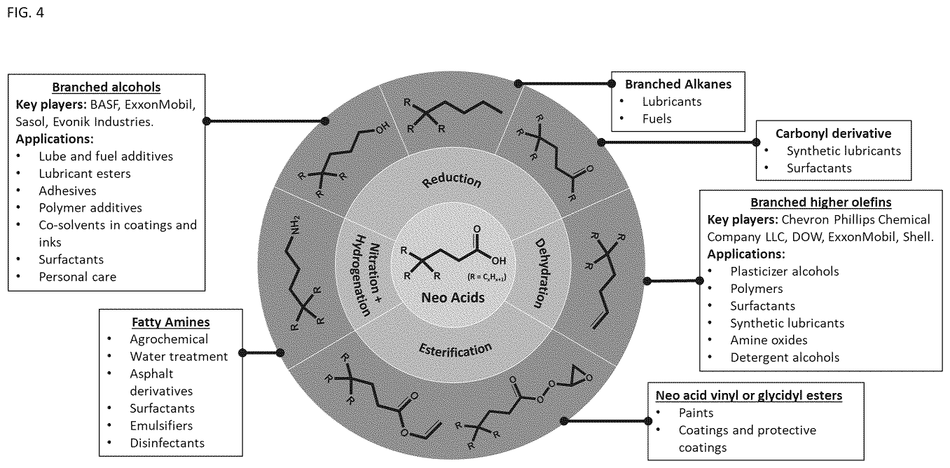

The neo acids and derivatives thereof of the present invention may be used to make a wide range of products, including lubricants, polymers, adhesives, agrochemicals, paints, coatings, protective coatings, construction, personal care, or cosmetics (FIG. 4). For example, these compounds may be used as building blocks to make lubricants, polymers, plastics, rubber, adhesives, sealants, agrochemicals, paints, coatings, protective coatings, construction, technical ceramics, personal care, or cosmetics. The derivatives may be branched alcohols obtainable through reduction with applications as lubricant and fuel additives, lubricant esters adhesives, polymer additives, co-solvent in coatings and inks, surfactants, or personal care products; branched alkanes obtainable through reduction with applications as lubricants and fuels; carbonyls obtainable through reduction with applications as synthetic lubricants and surfactants; fatty amines obtainable through nitration and hydrogenation with applications as agrochemicals, water treatment, asphalt derivatives, surfactants, emulsifier, and disinfectants; branched higher olefins obtainable through dehydration with applications as plasticizer alcohols, polymers, surfactants, synthetic lubricants, amine oxides, and detergent alcohols; or neo acid vinyl or glycidyl esters obtainable through esterification with applications as paints, coatings, and protective coatings.

The present invention provides a method for producing a neo acid (NA). The NA production method comprises hydrodeoxygenation (HDO) of a furan-containing neo acid (FNA) in the presence a metal triflate and a hydrogenation catalyst. The metal triflates may be selected from the group consisting of Ag(OTf), Zn(OTf)₂, Sn(OTf)₂, Cu(OTf)₂, Sc(OTf)₃, Nd(OTf)₃, Eu(OTf)₃, Sm(OTf)₃, La(OTf)₃, Al(OTf)₃, Ce(OTf)₄, Hf(OTf)₄, Zr(OTf)₄, and W(OTf)₆. The hydrogenation catalyst may be a metal based catalyst. The metal based catalyst may be selected from the group consisting of metal catalysts supported on carbon or acidic materials and nickel-based catalysts. The metal catalysts may be selected from the group consisting of Pd/C, Pd/SiO₂ and Pd/Al₂O₃. The nickel-based catalysts may be Raney Ni. The FNA may have a bio-based content in the range of 20-100%, 30-100%, 40-100%, 50-100%, 60-100%, 70-100%, 80-100%, 90-100%, 20-90%, 30-90%, 40-90%, 50-90%, 60-90%, 70-90% or 80-90%, according to, for example, ASTM-D6866.

The NA production method may further comprise hydroxyalkylation/alkylation (HAA) of 2-alkylfuran with levulinic acid or pyruvic acid in the presence of an acid catalyst, for example, a Brønsted acid catalyst. The acid catalyst may be selected from the group consisting of liquid acids and solid acids. The liquid acids may be selected from the group consisting of inorganic liquid acids, organic liquid acids and combinations thereof. The solid acid catalysts may be selected from the group consisting of sulfonic acid resins, sulfonic acid functionalized cross lined polystyrene resins, microporous acid zeolite and mesoporous aluminosilicate. At least one of 2-alkylfuran, levulinic acid and pyruvic acid may be from a renewable carbon source. The renewable carbon source may be non-food biomass. As a result, a furan-containing neo acid (FNA) is produced.

According to the neo acid production method, the neo acid (NA) may have a bio-based content in the range of 20-100%, 30-100%, 40-100%, 50-100%, 60-100%, 70-100%, 80-100%, 90-100%, 20-90%, 30-90%, 40-90%, 50-90%, 60-90%, 70-90% or 80-90%, according to, for example, ASTM-D6866.

According to the neo acid production method, the resulting neo acid (NA) has a structure of any one of formulae 9-12, and the furan-containing neo acid (FNA) may have a structure of any one of formulae 1-4.

The present invention provides a method for producing a branched alkane (BA). The BA production method comprises hydrodeoxygenation (HDO) of a furan-containing neo acid (FNA) in the presence of any suitable hydrodeoxygenation catalyst, such as a solid acid supported metal-metal oxide catalyst or a physical mixture of a metal-based catalyst with a solid acid. The solid acid supported metal-based catalyst may be selected from the group consisting of Ni/ZSM-5, Pd/ZSM-5, Pd/BEA, and a combination thereof. The physical mixture of a metal-based catalyst with a solid acid may be Pd/C+ZSM-5, Pd/C+BEA, or Pt/C+BEA. The supported metal-metal oxide catalyst may be Ir-ReOx/SiO₂, Ir—MoOX/SiO₂ or 1M2MO/SiO₂, wherein 1M=Ir, Ru, Ni, Co, Pd, Pt, or Rh and 2M=Re, Mo, W, Nb, Mn, V, Ce, Cr, Zn, Co, Y, or Al. The FNA may have a bio-based content in the range of 20-100%, 30-100%, 40-100%, 50-100%, 60-100%, 70-100%, 80-100%, 90-100%, 20-90%, 30-90%, 40-90%, 50-90%, 60-90%, 70-90% or 80-90%, according to, for example, ASTM-D6866.

The BA production method may further comprise hydroxyalkylation/alkylation (HAA) of 2-alkylfuran with levulinic acid or pyruvic acid in the presence of an acid catalyst, for example, a Brønsted acid catalyst. The acid catalyst may be selected from the group consisting of liquid acids and solid acids. The liquid acids may be selected from the group consisting of inorganic liquid acids, organic liquid acids and combinations thereof. The solid acid catalysts may be selected from the group consisting of sulfonic acid resins, sulfonic acid functionalized cross lined polystyrene resins, microporous acid zeolite and mesoporous aluminosilicate. At least one of 2-alkylfuran, levulinic acid and pyruvic acid may be from a renewable carbon source. The renewable carbon source may be non-food biomass. As a result, a furan-containing neo acid (FNA) is produced.

According to the BA production method, the BA may have a bio-based content in the range of 20-100%, 30-100%, 40-100%, 50-100%, 60-100%, 70-100%, 80-100%, 90-100%, 20-90%, 30-90%, 40-90%, 50-90%, 60-90%, 70-90% or 80-90%, according to, for example, ASTM-D6866.

According to the BA production method, the BA may have a structure of any one of formulae 17-20, and the FNA may have a structure of any one of formulae 1-4.

The present invention provides a method for producing a tetrahydrofuran-containing neo acid (THFNA). The THFNA production method comprises hydrogenation of a furan-containing neo acid (FNA) in the presence of a hydrogenation catalyst. The hydrogenation catalyst may be a metal based catalyst. The metal based catalyst may be selected from the group consisting of palladium catalysts supported on carbon or acidic materials and nickel-based catalysts. The palladium catalysts may be selected from the group consisting of Pd/C, Pd/SiO₂ and Pd/Al₂O₃. The nickel-based catalysts may be Raney Ni. The FNA may have a bio-based content in the range of 20-100%, 30-100%, 40-100%, 50-100%, 60-100%, 70-100%, 80-100%, 90-100%, 20-90%, 30-90%, 40-90%, 50-90%, 60-90%, 70-90% or 80-90%, according to, for example, ASTM-D6866.

The THFNA production method may further comprise hydroxyalkylation/alkylation (HAA) of 2-alkylfuran with levulinic acid or pyruvic acid in the presence of an acid catalyst, for example, a Brønsted acid catalyst. The acid catalyst may be selected from the group consisting of liquid acids and solid acids. The liquid acids may be inorganic liquid acids, organic liquid acids or a combination thereof.

The solid acid catalysts may be selected from the group consisting of sulfonic acid resins, sulfonic acid functionalized cross lined polystyrene resins, microporous acid zeolite and mesoporous aluminosilicate. At least one of 2-alkylfuran, levulinic acid and pyruvic acid may be from a renewable carbon source. The renewable carbon source may be non-food biomass. As a result, a furan-containing neo acid (FNA) is produced.

According to the THFNA production method, the tetrahydrofuran-containing neo acid (THFNA) may be a bio-based content in the range of 20-100%, 30-100%, 40-100%, 50-100%, 60-100%, 70-100%, 80-100%, 90-100%, 20-90%, 30-90%, 40-90%, 50-90%, 60-90%, 70-90% or 80-90%, according to, for example, ASTM-D6866.

According to the THFNA production method, the tertrahydrofuran-containing neo acid (THFNA) may have a structure of any one of formulae 21-24, and the furan-containing neo acid (FNA) may have a structure of any one of formulae 1-4.

The present invention provides a method for producing a furan-containing neo ester (FNE). The FNE production method comprises esterification of the FNA with an alcohol. The esterification may be performed with or without the presence of an acid catalyst. The acid catalyst may be selected from the group consisting of liquid acids and solid acids. The liquid acid may be selected from the group consisting of inorganic liquid acids, organic liquid acids and a combination thereof. The solid acid catalysts may be selected from the group consisting of sulfonic acid resins, sulfonic acid functionalized cross lined polystyrene resins, microporous acid zeolite and mesoporous aluminosilicate. The FNA may have a bio-based content in the range of 20-100%, 30-100%, 40-100%, 50-100%, 60-100%, 70-100%, 80-100%, 90-100%, 20-90%, 30-90%, 40-90%, 50-90%, 60-90%, 70-90% or 80-90%, according to, for example, ASTM-D6866.

The FNE production method may further comprise hydroxyalkylation/alkylation (HAA) of 2-alkylfuran with levulinic acid or pyruvic acid in the presence of an acid catalyst, for example, a Brønsted acid catalyst. The acid catalyst may be selected from the group consisting of liquid acids and solid acids. The liquid acid may be selected from the group consisting of inorganic liquid acids, organic liquid acids and a combination thereof. The solid acid catalysts may be selected from the group consisting of sulfonic acid resins, sulfonic acid functionalized cross lined polystyrene resins, microporous acid zeolite and mesoporous aluminosilicate. At least one of 2-alkylfuran, levulinic acid and pyruvic acid may be from a renewable carbon source. The renewable carbon source may be non-food biomass. As a result, a furan-containing neo acid (FNA) is produced.

According to the FNE production method, the FNE may have a bio-based content in the range of 20-100%, 30-100%, 40-100%, 50-100%, 60-100%, 70-100%, 80-100%, 90-100%, 20-90%, 30-90%, 40-90%, 50-90%, 60-90%, 70-90% or 80-90%, according to, for example, ASTM-D6866.

According to the FNE production method, the furan-containing neo ester (FNE) may have a structure of any one of formulae 25-28, and the furan-containing neo acid (FNA) has a structure of any one of formulae 1-4.

The term “about” as used herein when referring to a measurable value such as an amount, a percentage, and the like, is meant to encompass variations of +20% or +10%, more preferably +5%, even more preferably 1%, and still more preferably +0.1% from the specified value, as such variations are appropriate.

### Example 1. Production of Neo Acids and Derivatives Thereof

### A. Materials

Aquivion® PW79S (coarse powder, Brunauer-Emmett-Teller (BET) surface area <1 m²/g, and 1.26 mmol H+/g), Aquivion® PW98 (coarse powder, BET surface area <1 m²/g, and 1.0 mmol H+/g), phosphotungstic acid hydrate (BET surface area <1 m²/g and 1.04 mmol H+/g), phosphomolybdic acid hydrate (BET surface area 1-5 m²/g and 1.5 mmol H+/g), amorphous silica alumina (ASA; catalyst support grade 135; 12 wt % Al₂O₃; >90% AS-100 mesh; pore size, 5.4 nm; BET surface area, 569 m²/g; and 0.34 mmol H+/g), 2-methylfuran (99%), 2-pentylfuran (≥98.0%), 2-ethylfuran (≥99.0%), levulinic acid (98%), pyruvic acid (98%), eicosane (99%), ethyl acetate (99.8%), acetic acid (≥99.7%), methanesulfonic acid (≥99.0%), p-toluenesulfonic acid monohydrate (≥98.5%), triflic acid (≥99.0%), Eu(OTf)₃ (98%), La(OTf)₃ (99%), Nd(OTf)₃ (98%), Sc(OTf)₃ (99%), Cu(OTf)₂ (99%), Zn(OTf)₂ (99%), Ag(OTf) (≥98%), Sm(OTf)₃ (98%), Pd/C (10 wt % Pd loading), Pt/C (10 wt % Pt loading), pyridine (99.8%), and N,O-Bis(trimethylsilyl)trifluoroacetamide (BSTFA) (≥99.0%) were purchased from Sigma-Aldrich. Cyclohexane (99.9%) and methanol (≥99.9%) was purchased from Fisher Chemical. 2-Propylfuran (>98%) and 2-butylfuran (>98%) were purchased from Tokyo Chemical Industry Co. Ltd. HY (CBV720; Si/Al=15; pore size, ˜0.7 nm; BET surface area 780 m²/g; and 0.31 mmol H+/g) was purchased from Zeolyst. H₂SO₄ (5 M) was purchased from Fluka. Ru/C (10 wt % Ru loading) was purchased from Riogen. Hf(OTf)₄, Al(OTf)₃ (99%), Ce(OTf)₄ (98%), Sn(OTf)₂ (97%), and 2-heptylfuran (97%) with the purity given in the parentheses were purchased from Alfa Aesar.

### B. Material Pretreatment

2-Alkylfurans, including 2-methylfuran, 2-ethylfuran, 2-propylfuran, 2-butylfuran, 2-pentylfuran, 2-hexylfuran and 2-heptylfuran, were purified by vacuum distillation for use in the examples below.

### C. Reaction Procedures

### 1. Hydroxyalkylation/Alkylation (HAA) Reaction to Make Furan-Containing Neo Acid (FNA)

In a typical reaction, 20 mmol (2.76 g) 2-alkylfuran, 10 mmol (1.16 g) levulinic acid or 10 mmol (0.88 g) pyruvic acid, and a calculated amount of catalyst were mixed in a 50 mL round-bottom flask without any solvent. The flask was placed in a preheated oil bath and magnetically stirred at 800 rpm. The reaction was run at the desired temperature for a specified reaction period. After the reaction, the solution was diluted using 10 ml of cyclohexane and 5 mL ethyl acetate solvents. Ethyl acetate was used to solubilize unreacted levulinic acid. Eicosane (C₂₀) was added as an internal standard, and the catalyst was separated from the solution by syringe filtration.

For the catalyst recyclability experiments, the liquid product was decanted out after each cycle. The remaining catalyst was washed thrice with cyclohexane and ethyl acetate to remove surface-adsorbed unreacted reactants and products. Then, dried in a vacuum oven at 60° C. for 1 h before reuse in the next cycle.

### 2. Hydrodeoxygenation (HDO) Reaction to Make Neo Acid (NA)

HDO of FNA over metal triflate was performed in a 50-mL Parr reactor (4790 pressure vessel, Parr Instrument Company) with an inserted glass liner and a magnetic stirrer. In a typical reaction, 1 mmol (0.37 g) of FNA, 6 mol % (0.029 g) Al(OTf)₃, 2 mol % (0.021 g) of 10 wt % Pd/C, and solvent (10 mL of cyclohexane) were added to the reactor, and the reactor was sealed with the reactor head equipped with a thermocouple, a rupture disk, a pressure gauge, and a gas release valve. The reactor was purged with 1 MPa N₂ for five times and followed by 1 MPa H₂, and finally pressurized to the desired H₂ pressure. The reaction mixture was heated to the desired temperature with continuous stirring at 750 rpm. The heating time to reach the set temperature was about 20 min. Once the desired temperature was reached, the mixture was run for a specified reaction period. Upon completion, the reactor was immediately transferred to an ice bath, cooled to room temperature, and H₂ was released. The reaction solution was diluted using 5 mL of ethyl acetate with a small amount of decane (Cio) as an internal standard, and the catalyst was separated from the solution by filtration.

3. Hydrogenation Reaction to Make Tetrahydrofuran-Containing Neo Acid (THFNA) Hydrogenation of FNA was carried out in a 50-mL Parr reactor (4790 pressure vessel, Parr Instrument Company) with an inserted glass liner and a magnetic stirrer. First, Pd/C catalyst was pretreated at 200° C. with a temperature ramp of 10° C./min for 1 hour with H₂ (50 mL/min). Then, Pd/C catalyst (0.03 g), FNA (0.5 g), and 10 ml of cyclohexane were added to the reactor. The reactor was sealed with the reactor head equipped with a thermocouple, a rupture disk, a pressure gauge, and a gas release valve. The reactor was purged with 1 MPa N₂ five times and followed by 1 MPa H₂ five times, and finally pressurized to the desired H₂ pressure of 6 MPa H₂. The reaction mixture was heated to 60° C. with continuous stirring at 750 rpm. The heating time to reach the set temperature was about 5 min. Once the desired temperature was reached, the mixture was run for 1 h. Upon completion, the reactor was immediately transferred to an ice bath, cooled to room temperature, and H₂ was released. The reaction solution was diluted using 5 mL of ethyl acetate with a small amount of decane (Cio) as an internal standard, and the catalyst was separated from the solution by filtration.

### 4. Process of Making an Alkane Compound by Hydrodeoxygenation (HDO) Reaction

HDO of FNA over Ir—ReOₓ/SiO₂ was performed in a 50-mL Parr reactor with an inserted glass liner and a magnetic stirrer. Ir—ReOₓ/SiO₂ was prepared according to the literature (Liu et al., Science advances, 5(2), p.eaav5487 (2019)). In a typical reaction, the catalyst (0.15 g) and solvent (10 ml of cyclohexane) were added to the reactor for catalyst prereduction, and the reactor was sealed with the reactor head equipped with a thermocouple, a rupture disk, a pressure gauge, and a gas release valve. The mixture was heated at 200° C. and 5 MPa H₂ for 1 hour at 250 rpm. Upon prereduction, the reactor was cooled to room temperature and H₂ was released. Then, we added FNA (0.3 g), closed the reactor head immediately, purged the reactor with 1 MPa H₂ for five times, pressurized to 5 MPa H₂, and heated the reaction mixture to the desired temperature with continuous stirring at 500 rpm. Upon reaction, the reactor was cooled and depressurized. The reaction solution was diluted using 15 mL of cyclohexane with a small amount of eicosane as an internal standard, and the catalyst was separated from the solution by filtration.

### 5. Product Analysis by Derivatization

Silylation, a derivatization technique, was implemented to improve the chromatographic behavior of the polar neo acid compounds and intermediates. Silylation works by selectively replacing the active hydrogens on the compound with an alkylsilyl group, resulting in less polar and more volatile compounds. Hence, detection and better separation on gas chromatography (GC). In a typical reaction, 100 μL product, 900 μL solvent, 250 μL pyridine, and 250 μL N,O-Bis(trimethylsilyl)trifluoroacetamide (BSTFA) were mixed in a GC vial. Then, the solution was heated on a hot plate at 65° C. for 20 minutes.

### 6. Analysis of Products

The products were analyzed using a gas chromatograph (GC, Agilent 7890A) equipped with a HP-1 column and a flame ionization detector using eicosane (C₂₀) as an internal standard. The products were identified by a GC (Agilent 7890B) mass spectrometer (MS) (Agilent 5977A with a triple-axis detector) equipped with a DB-5 column, high-resolution MS with liquid injection field desorption ionization.

The conversion and the yield of all products were calculated on a carbon molar basis as follows:

$$Conversion ( \% ) = \frac{moles of initial reactant - moles of unreacted reactant}{moles of initial reactant} \times 100 \% \\ \\ Yield ( \% ) = \frac{{moles}_{product} \times C atoms in product}{moles of total C atoms of initial reactants} \times 100 \%$$

### D. Results

### 1. Hydroxyalkylation/Alkylation (HAA) to Make Furan-Containing Neo Acid (FNA)

a. Initial Results and Catalyst Screening

Initial experiments over Aquivion PW79S catalyst resulted in 52.5% yield of C23 HAA product (C₂₃H₄₆₀₃, FNA) at 64% and 59.7% conversion of 2-PF and LA respectively Small fractions of other products form via ring-opening of HAA product (C₂₃H₃₆O₅, FNA RO) and self-condensation of 2-PF (C₂₇H₄₂O₃, SC-1; C₂₇H₄₄₀₄, SC-2; C₃₆H₅₆O₄, SC-3; C₃₆H₅₄O₃, SC-4) referred here to as PFSCs (2-pentylfuran self-condensation products) (FIG. 5). Blank experiments with 2-PF and levulinic acid alone confirmed that no other form of 2-PF self-condensation products were formed, and no self-condensation of LA occurred. All products had >20 carbon atoms and a branched backbone. The FNA RO was converted to C₂₃-NA upon selective HDO.

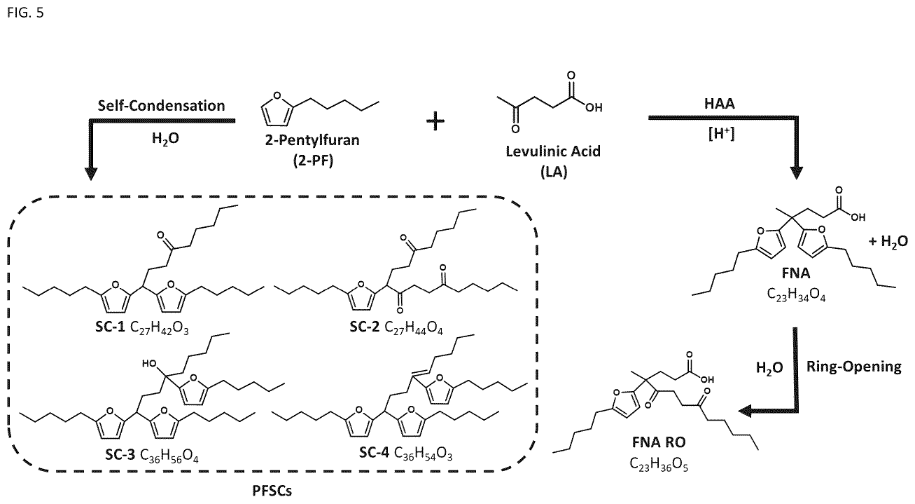

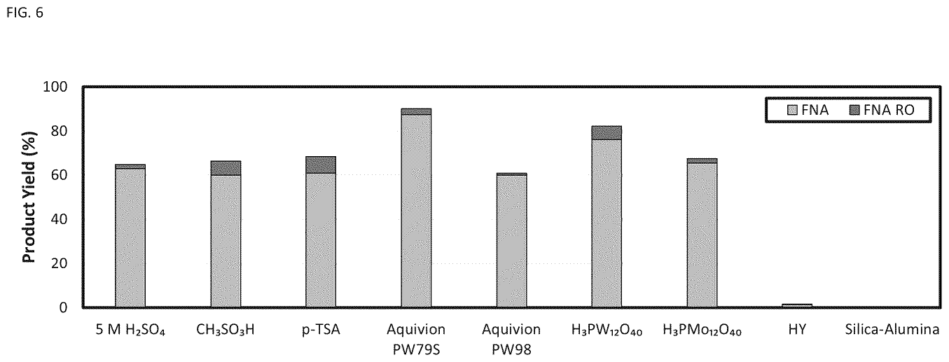

Several homogeneous and heterogeneous acid catalysts were screened under the initial reaction conditions and found that Aquivion PW79S yields the highest FNA (FIG. 6). This is attributed to its high acid density and strength of the catalyst. Microporous acid zeolite and mesoporous aluminosilicate were also screened, and they did not show any activity.

b. Multi-Parameter Optimization Using NEXTorch

To optimize 2-pentylfuran and levulinic acid conversion and product yield the multi-parameter optimization was conducted using NEXTorch, an active learning-driven optimization toolkit. An initial sampling of 16 points from a Latin hypercube design varying temperature, reaction time, molar ratio, and catalyst loading was performed. NEXTorch then process the data and predict optima where 4 subsequent sampling points generated per iterations.

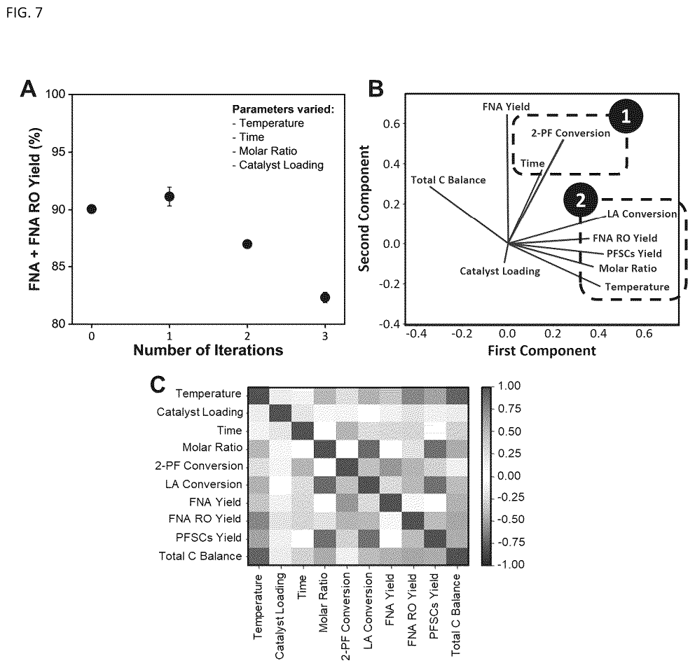

The yield of FNA and FNARO increased between iteration 0 and 1, but decreased in the third and fourth iterations, suggesting an exploration with no increased product (FIG. 7A). This process highlights the utility of a data driven approach for optimizing product yield. If a traditional central composite design with four factors was used, a total of 80 experiments would have been needed which might still not cover the true optimum. With NEXTorch, the experimental time (<30 runs to identify an optimum) and consumables were reduced efficiently.

At the optimum reaction condition, nearly 15% of the levulinic acid remains unconverted. Thus, with the data obtained from multi-parameter optimization a correlation matrix was generated to understand the interactions between different reaction variables (FIGS. 7B-C). The results showed that high LA conversion can be obtained at higher temperature and molar ratio. However, the high conversion is due to formation of a side product angelica lactones but not due to the participation of LA in the desired HAA reaction. This is supported by the negative correlation between LA conversion and the total carbon balance.

2-PF conversion is positively correlated with time (cluster 1, FIG. 7B), LA conversion, FNA RO yield, PFSCs yield, molar ratio, and temperature are positively correlated (cluster 2, FIG. 7B), FNA yield is inversely correlated with temperature, and temperature is inversely correlated (strongly) to total carbon balance, as well as the rest of cluster 2 (weakly).

Correlation matrix agree with the principle component analysis (PCA) results. Strong positive correlation was found for the molar ratio and 2-pentylfuran self-condensation product yield, molar ratio and LA conversion, temperature and FNA RO yield. Strong negative correlation was found for the reaction temperature and total carbon balance

With that, under the optimum reaction conditions, an overall 90% yield of FNA and FNA RO was achieved.

c. Recyclability of Aquivion PW79S

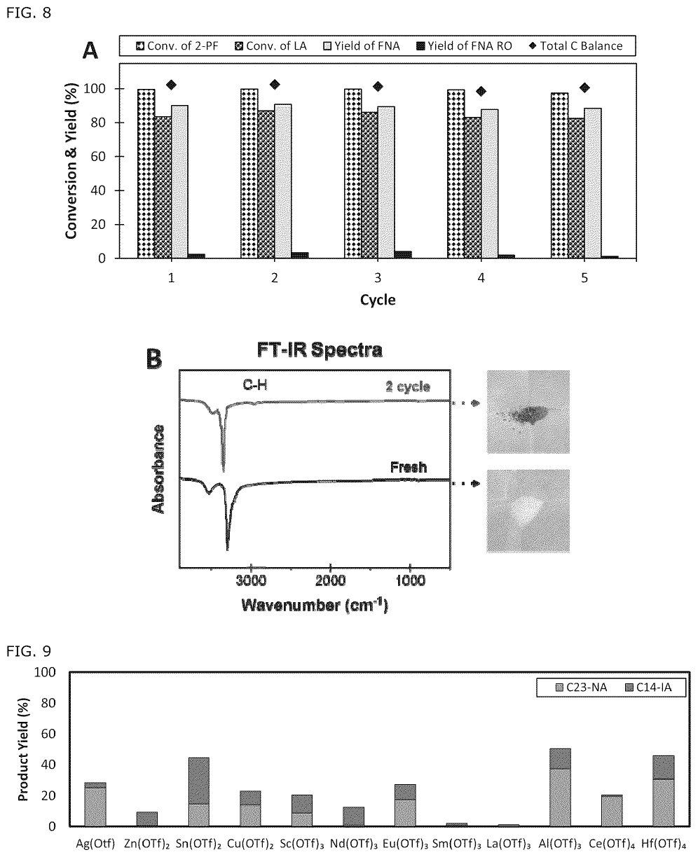

Under the optimized reaction conditions, the catalyst was successfully recycled for the five consecutive cycles without significant loss of activity (FIG. 8A). The slight difference in LA conversion and product yield between each cycle can be attributed to the small deactivation of the catalyst, likely due to coverage of the active sites by adsorbed products. This is supported by the FT-IR results (FIG. 8B) where the additional bands between 2800-3000 cm⁻¹ were detected which corresponds to the C—H stretching.

d. Extending the Chemistry

Table 2 shows synthesis of FNA and FNA RO using 2-alkylfurans and keto acid of varying molecular sizes under the optimized reaction conditions of 20 mmol 2-Alkylfuran, (2-Alkylfuran/Keto acid) (mol/mol)=2, 0.107 mmol H+aquivion PW79S, 6 hours, 65° C., 800 rpm.

TABLE 2
Synthesis of a series of FNA and FNA RO using 2-alkylfurans and keto
acid of varying alkyl chain lengths.

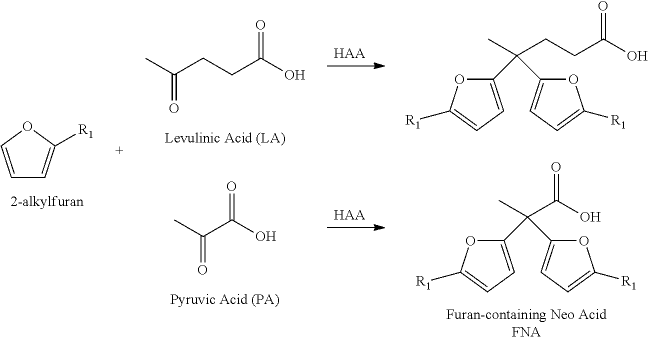

| | Reagents | | | Yield FNA & | Total C | % |
|---|---|---|---|---|---|---|
| Entry | R₁ | Keto Acid | #C | FNA RO (%) | Balance (%) | Error |
| 1 | Methyl | LA | C15 | 47.3 | 75.1 | 0.9 |
| 2 | Ethyl | LA | C17 | 76.7 | 94.4 | 1.2 |
| 3 | n-Propyl | LA | C19 | 86.9 | 101.9 | 0.6 |
| 4 | n-Butyl | LA | C21 | 74.9 | 87.1 | 2.0 |
| 5 | n-Pentyl | LA | C23 | 90.1 | 100.9 | 0.1 |
| 6 | n-Heptyl* | LA | C27 | 82.0 | 89.4 | 0.2 |
| 7 | Methyl | PA | C13 | 85.6 | 93.2 | 0.4 |
| 8 | Ethyl | PA | C15 | 87.2 | 89.6 | 2.9 |
| 9 | n-Propyl | PA | C17 | 91.9 | 95.1 | 0.4 |
| 10 | n-Butyl | PA | C19 | 89.6 | 92.2 | 0.9 |
| 11 | n-Pentyl | PA | C21 | 85.5 | 89.0 | 2.5 |
| 12 | n-Heptyl | PA | C25 | 79.2 | 84.0 | 0.2 |

### 2. Hydrodeoxygenation (HDO) to Make Neo Acid (NA)

a. Catalyst screening
   i. Metal triflate screening with 10 wt % Pd/C

Preliminary HDO experiments were performed following the literature conditions (Dutta et al., ACS Catalysis, 7(8), 5491-5499 (2017)) using metal triflate and Pd/C catalysts. It showed successful conversion of the HAA product into C₂₃-neo acid (C₂₃-NA) (FIG. 9) but surprisingly product yield was low at nearly complete conversion of the reactant. In this HDO chemistry Pd/C acts as a hydrogenation catalyst and metal triflate acts as the ring-opening catalyst. Undesirable side reactions resulted in the formation of a cracked product, C₁₄-iso acid (C₁₄-IA), and high molecular weight products that were not detected by GC/GCMS. Although iso acid is a side product in this reaction, it could be valuable for the industry. Hence, its yield was accounted in our studies. Then, several other metal triflates were screened and found that aluminum triflate is effective resulting in highest neo acid yield (FIG. 9).

ii. Hydrogenation Catalyst Screening

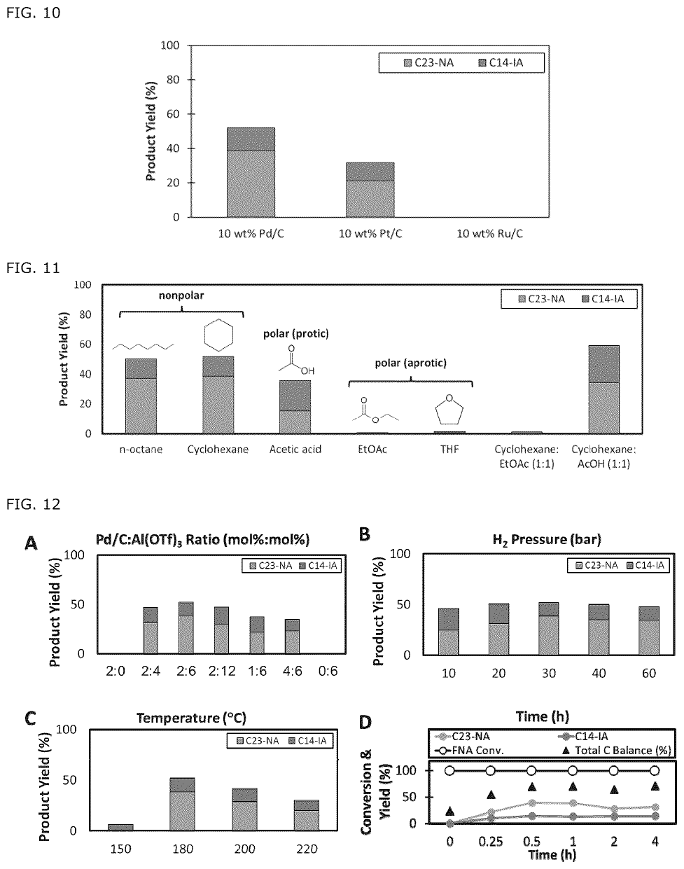

Hydrogenation catalysts were screened for the preparation of neo acid from FNA and FNA RO under the reaction conditions of 1 mmol FNA, 6 mol % Al(OTf)₃, 2 mol % hydrogenation catalyst, 10 mL n-octane, 30 bar H₂, 180° C., 1 h, 500 rpm. The Pd/C hydrogenation catalyst resulted in highest neo acid yield (37%) at complete conversion of the reactants with 70% total carbon balance (FIG. 10).

b. Solvent Screening

Next, the effect of organic solvent on the HDO reaction was studied (FIG. 11) and found that non-polar organic solvents yield higher C₂₃-NA due to the better solubility of reactants, intermediates, and products as well as they do not interact with the metal triflate. When acetic acid, a polar protic organic solvent was used, formation of cracked products and esters was observed along with the neo acid. In another attempt a mixture of polar and non-polar organic solvents was explored and no improvement in C₂₃-NA yield was observed.

Even though both n-octane and cyclohexane organic solvents resulted in higher NA yield, cyclohexane was selected for the further optimizations due to its lower boiling point.

c. Reaction Parameter Optimization

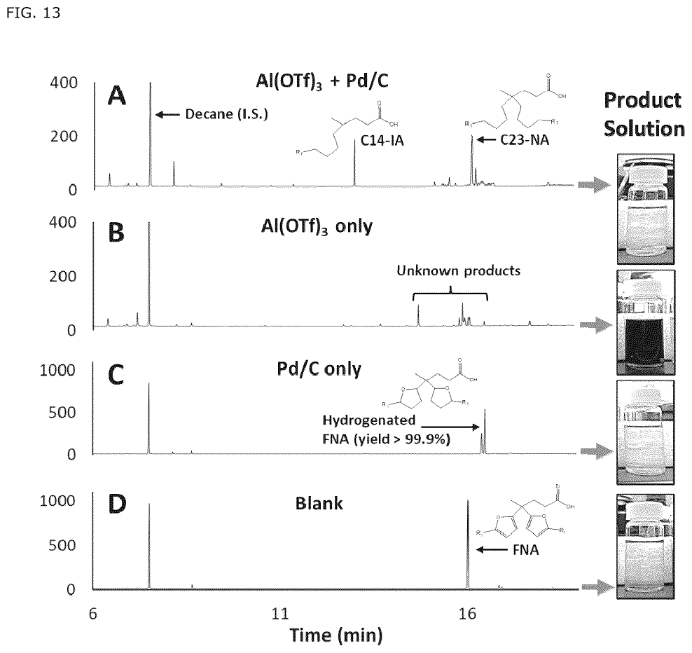

To investigate the role of hydrogenation catalyst and metal triflate on the HDO chemistry, experiments were conducted with Pd/C alone, metal triflate alone and without any of the catalysts and results were compared with optimum condition results (Table 3). The GC chromatograms for each reaction are overlaid in FIG. 13. These results indicate that both hydrogenation catalyst (Pd/C) and metal triflate are required to form neo acid.

TABLE 1
The effect of catalysts on product distribution.

| | FNA + FNA RO | C23-NA | C14-IA | Hydrogenated |
|---|---|---|---|---|
| Catalyst(s) | Conversion (%) | yield (%) | yield (%) | FNA yield (%) |
| Al(OTf)₃ + | 100 | 40 | 15 | 0 |
| Pd/C | | | | |
| Al(OTf)₃ | 91 | 0 | 0 | 0 |
| only | | | | |
| Pd/C only | 100 | 0 | 0 | 100 |
| Blank (no | 0 | 0 | 0 | 0 |
| catalysts) | | | | |

Then the effect of varying catalyst molar ratio, hydrogen pressure, temperature, and time were studied (FIG. 12).

For catalyst molar ratio variation, it was found that the HDO reaction required a suitable ratio of the hydrogenation catalyst (Pd/C) and metal triflate to form neo acid (FIG. 12A)

For pressure variation experiments, it was found that C₂₃-NA yield increases with hydrogen pressure where it plateaus after 30 bar. The increase of hydrogen pressure favors the adsorption of Pd/C on hydrogen, ensuring complete hydrogenation of the intermediates and final products, minimizing any undesired side reactions, and thus improving yield of C₂₃-NA (FIG. 12B).

For temperature variation, under all the reaction temperatures explored (150-220° C.), the reactant was completely converted and with increasing reaction temperature the yield of neo acid increased firstly and then decreased (FIG. 12C). This suggests that at higher temperatures, the neo acid might be participating in the side reactions forming high molecular weight products that were not detected by GC/GCMS resulting in lower total carbon balance.

Lastly, the time-dependent study was conducted. At time zero, which is the pre-heating time, all the reactant was converted however no C₂₃-NA was detected. As the reaction proceeds, C₂₃-NA starts to form but plateaued after 30 minutes (FIG. 12D). Further investigations showed that the plateaued NA yield is due to the side reactions by the multiple functional groups (—OH, —COOH, C═C, C—O—C) involved in the reactant, intermediate and possible side products.

With the optimization studies, a 40% yield of C₂₃-NA and 15% yield of C14-IA was achieved at Pd/C:Al(OTf)₃ ratio of 2:6, 30 bar H₂, 180° C., and 30 minutes.

### Example 2. Other Derivatives

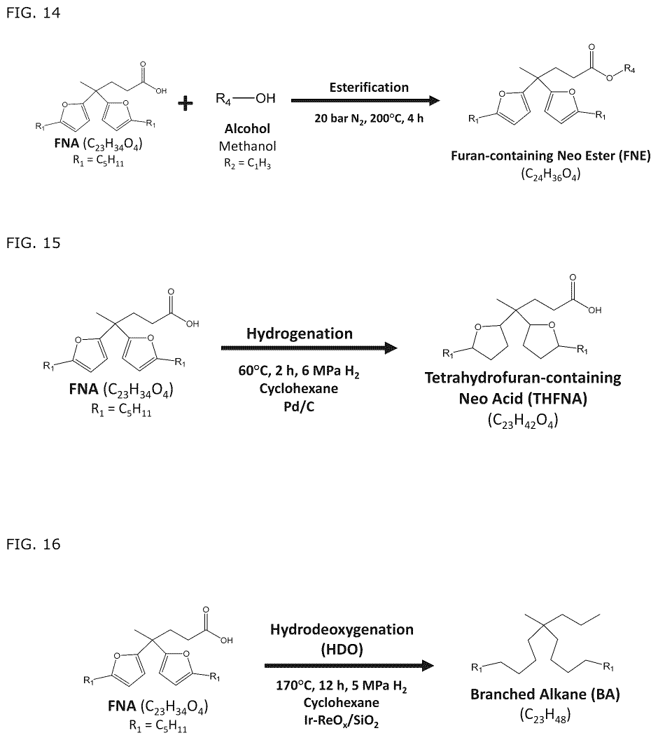

A furan-containing neo ester (FNE) was made by esterification of FNA with alcohol (methanol) (Scheme 4) (FIG. 14).

The FNA conversion and FNE yield upon esterification of FNA with alcohol were studied under the reaction conditions of 4 g HAA reaction product, 16 mL methanol, 20 bar N₂, 200° C., 4 h and achieved 93.5% FNE at complete conversion of FNA.

A tetrahydrofuran-containing neo acid (THFNA) was made by hydrogenation of FNA (Scheme 5) (FIG. 15) under the reaction conditions of 0.5 g HAA reaction product, 0.03 Pd/C, 10 mL cyclohexane, 60° C., 2 h, 6 MPa H₂, and 500 rpm. The Pd/C was pre-reduced under H₂ (50 mL/min) at 200° C. for 1 hr. The THFNA yield of 94% was obtained at complete conversion of FNA.

A branched alkane (BA) was made by hydrodeoxygenation of FNA (Scheme 6) (FIG. 16) under the reaction conditions of 0.3 g HAA product, 0.15 g Ir—ReOₓ/SiO₂, 10 mL cyclohexane, 170° C., 12 h, 5 MPa H₂, and 500 rpm. The HDO reaction resulted in the formation of cracked product with C9-C21 chain length along with the C23 NA at complete conversion of the FNA (Table 4).

TABLE 4
Product distribution and total carbon balance for the mixture
of alkanes obtained from the HDO reaction of HAA product

| | Compound | | | | | | | |
|---|---|---|---|---|---|---|---|---|
| | | | | | | | | Total C |
| | C5 | C9 | C14 | C18 | C20 | C21 | C23 | Balance (%) |
| Yield | 23.2 | 20.3 | 23.8 | 0.8 | 4.5 | 14.0 | 6.7 | 93.4 |
| (%) | | | | | | | | |

Purification of the above compounds could be achieved by traditional product separation techniques such as vacuum distillation, extraction, and column chromatography.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. (Original) A compound having a structure of any one of formulae 1-28:

| Formula # | Structure | |
|---|---|---|
| 1 | 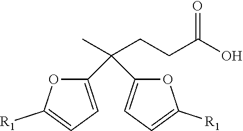 | R₁ is a C₂₋₁₈ alkyl group. |
| 2 | 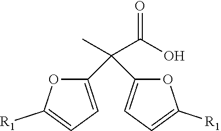 | Ri is a C₁₋₁₈ alkyl group. |
| 3 | 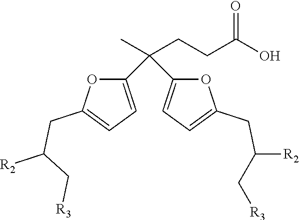 | R₂ and R₃ are independently selected from the group consisting of H and C₁₋₁₈ alkyl groups. |
| 4 | 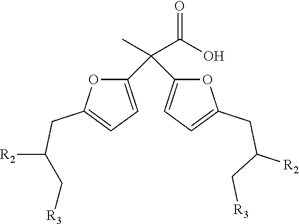 | R₂ and R₃ are independently selected from the group consisting of H and C₁₋₁₈ alkyl groups. |
| 5 | 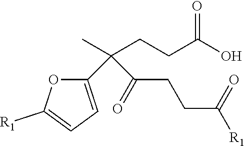 | R₁ is a C₁₋₁₈ alkyl group. |
| 6 | 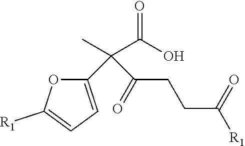 | R₁ is a C₁₋₁₈ alkyl group. |
| 7 | 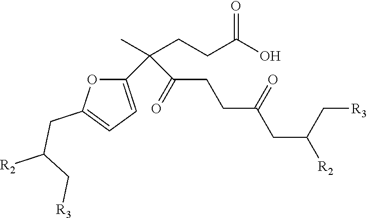 | R₂ and R₃ are independently selected from the group consisting of H and C₁₋₁₈ alkyl groups. |
| 8 | 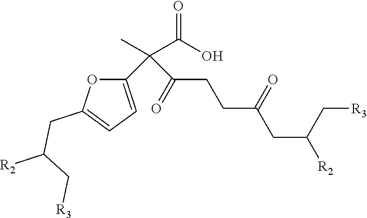 | R₂ and R₃ are independently selected from the group consisting of H and C₁₋₁₈ alkyl groups. |
| 9 | 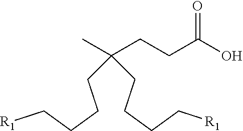 | R₁ is a C₁₋₁₈ alkyl group. |
| 10 | 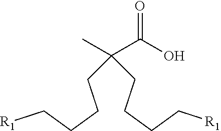 | R₁ is a C₁₋₁₈ alkyl group. |
| 11 | 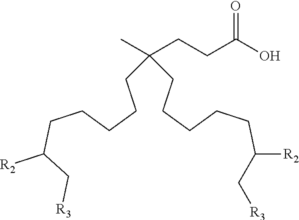 | R₂ and R₃ are independently selected from the group consisting of H and C₁₋₁₈ alkyl groups. |
| 12 | 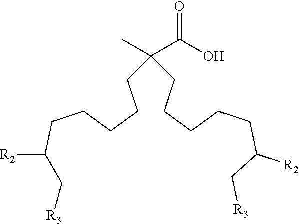 | R₂ and R₃ are independently selected from the group consisting of H and C₁₋₁₈ alkyl groups. |
| 13 | 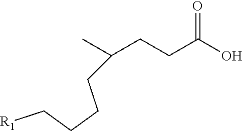 | R₁ is a C₁₋₁₈ alkyl group. |
| 14 | 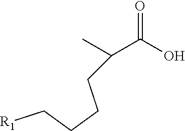 | R₁ is a C₁₋₁₈ alkyl group. |
| 15 | 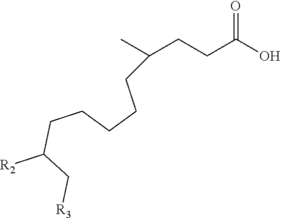 | R₂ and R₃ are independently selected from the group consisting of H and C₁₋₁₈ alkyl groups. |
| 16 | 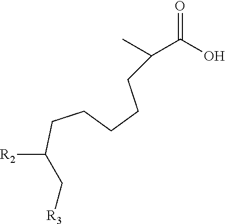 | R₂ and R₃ are independently selected from the group consisting of H and C₁₋₁₈ alkyl groups. |
| 17 | 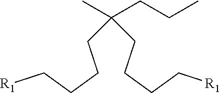 | R₁ is a C₁₋₁₈ alkyl group. |
| 18 | 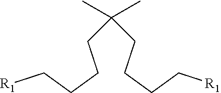 | Ri is a C₁₋₁₈ alkyl group. |
| 19 | 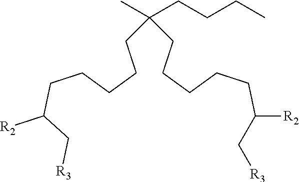 | R₂ and R₃ are independently selected from the group consisting of H and C₁₋₁₈ alkyl groups. |
| 20 | 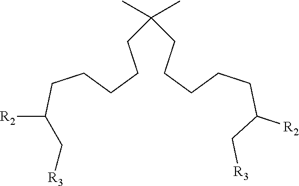 | R₂ and R₃ are independently selected from the group consisting of H and C₁₋₁₈ alkyl groups. |
| 21 | 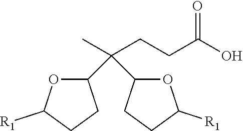 | R₁ is a C₁₋₁₈ alkyl group. |
| 22 | 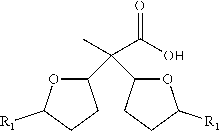 | R₁ is a C₁₋₁₈ alkyl group. |
| 23 | 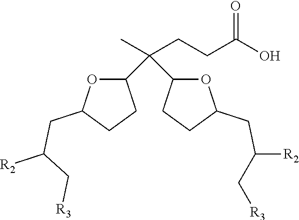 | R₂ and R₃ are independently selected from the group consisting of H and C₁₋₁₈ alkyl groups. |
| 24 | 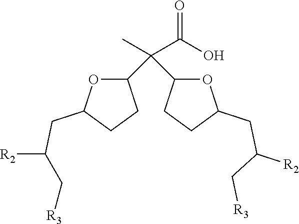 | R₂ and R₃ are independently selected from the group consisting of H and C₁₋₁₈ alkyl groups. |
| 25 | 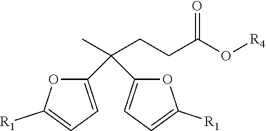 | R₁ is H or a C₁₋₁₈ alkyl group. R₄ is a C₃₋₁₈ alkyl group. |
| 26 | 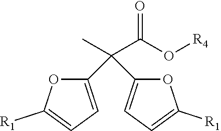 | R₁ and R₄ are independently a C₁₋₁₈ alkyl group. |
| 27 | 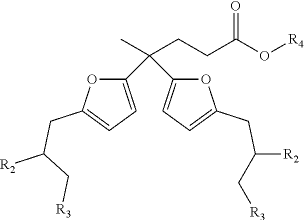 | R₂ and R₃ are independently selected from the group consisting of H and a C₁₋₁₈ alkyl group. R₄ is a C₃₋₁₈ alkyl group. |
| 28 | 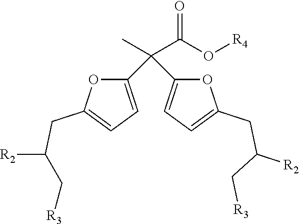 | R₂ and R₃ are independently selected from the group consisting of H and a C₁₋₁₈ alkyl group. R₄ is a C₃₋₁₈ alkyl group. |

2. The compound of claim 1, wherein the compound is a furan-containing neo acid (FNA) having a structure of any one of formulae 1-4.

3. The compound of claim 1, wherein the compound is a furan-containing neo acid ring opening (FNA RO) having a structure of any one of formulae 5-8.

4. The compound of claim 1, wherein the compound is a neo acid (NA) having a structure of any one of formulae 9-12.

5. The compound of claim 1, wherein the compound is an iso acid (IA) having a structure of any one of formulae 13-16.

6. The compound of claim 1, wherein the compound is a branched alkane (BA) having a structure of any one of formulae 17-20.

7. The compound of claim 1, wherein the compound is a tertrahydrofuran-containing neo acid (THFNA) having a structure of any one of formulae 21-24.

8. The compound of claim 1, wherein the compound is a furan-containing neo ester (FNE) having a structure of any one of formulae 25-28.

9. A method for producing a neo acid (NA), comprising hydrodeoxygenation (HDO) of a furan-containing neo acid (FNA) in the presence a metal triflate and a hydrogenation catalyst.

10. The method of claim 9, further comprising hydroxyalkylation/alkylation (HAA) of 2-alkylfuran with levulinic acid or pyruvic acid in the presence of an acid catalyst, wherein at least one of 2-alkylfuran, levulinic acid and pyruvic acid is from a renewable carbon source, whereby the furan-containing neo acid (FNA) is produced.

11. The method of claim 9, wherein the neo acid (NA) has a structure of any one of formulae 9-12.

12. The method of claim 9, wherein the furan-containing neo acid (FNA) has a structure of any one of formulae 1-4.

13. A method for producing a branched alkane (BA), comprising hydrodeoxygenation (HDO) of a furan-containing neo acid (FNA) in the presence of a solid acid supported metal-metal oxide catalyst or a physical mixture of a metal-based catalyst with a solid acid.

14. The method of claim 13, further comprising hydroxyalkylation/alkylation (HAA) of 2-alkylfuran with levulinic acid or pyruvic acid in the presence of an acid catalyst selected from the group consisting of liquid acids and solid acids, wherein at least one of 2-alkylfuran, levulinic acid and pyruvic acid is from a renewable carbon source, whereby the furan-containing neo acid (FNA) is produced.

15. The method of claim 13, wherein the branched alkane (BA) has a structure of any one of formulae 17-20.

16. The method of claim 13, wherein the furan-containing neo acid (FNA) has a structure of any one of formulae 1-4.

17. A method for producing a tetrahydrofuran-containing neo acid (THFNA), comprising hydrogenation of a furan-containing neo acid (FNA) in the presence of a hydrogenation catalyst.

18. The method of claim 17, further comprising hydroxyalkylation/alkylation (HAA) of 2-alkylfuran with levulinic acid or pyruvic acid in the presence of an acid catalyst, wherein at least one of 2-alkylfuran, levulinic acid and pyruvic acid is from a renewable carbon source, whereby the furan-containing neo acid (FNA) is produced.

19. (canceled)

20. The method of claim 17, wherein the furan-containing neo acid (FNA) has a structure of any one of formulae 1-4.

21. A method for producing a furan-containing neo ester (FNE), comprising esterification of the FNA with an alcohol.

22-24. (canceled)
